(19) United States
(12) Patent Application Publication
(10) Pub. No.: US 2004/0106632 A1
(43) Pub. Date: Jun. 3, 2004

(54) Salt of thiazolidinedione and its polymorphs as antidiabetic agents and method for obtaining them

(76) Inventors: Juan Carlos Del Castillo Nieto, Barcelona (ES); Francisco Marquillas Olondriz, Barcelona (ES); Elisabet De Ramon Amat, Barcelona (ES)

Correspondence Address: STEINBERG & RASKIN, P.C., 1140 AVENUE OF THE AMERICAS, 15th FLOOR, NEW YORK, NY, 10036-5803, US

(21) Appl. No.: 10/470,980

(22) PCT Filed: Jan. 21, 2002
(86) PCT No.: PCT/IB02/00229

(30) Foreign Application Priority Data
Jan. 31, 2001 (ES) P 200100273

(51) Int. Cl. A61K 31/513; C07D 417/02

(52) U.S. Cl. 514/269; 544/319

(57) ABSTRACT

This invention relates to a new salt of 5-(4-{2-′(6-methoxy-pyrimidin-4-yl)methyl-amino!-ethoxy}-benzyl)-thiazolidin-2,4-dione and its polymorphs which has high hypoglycemiant activity and which are therefore potentially useful in the treatment and/or prophylaxis of diabetes and/or other alterations or complications inherent to diabetes, such as hyyperglycemia or hyperlipidemia. This invention also relates to a method for making thereof.

### FIELD OF THE INVENTION

This invention relates to a new salt of thiazolidinedione and its polymorphs which has high hypoglycemiant activity and which are therefore potentially useful in the treatment and/or prophylaxis of diabetes and/or other alterations or complications inherent to diabetes, such as hyperglycemia or hyperlipidemia.

This invention also relates to a method for making said new salt of thiazolidinedione, together with its polymorphs.

### BACKGROUND OF THE INVENTION

Spanish patent application no. 9902533 disclosed compounds of thiazolidinedione which present high hypoglycemiant activity and which are therefore potentially useful in the treatment and/or prophylaxis of diabetes and/or other alterations or complications inherent to diabetes, such as hyperglycemia or hyperlipidemia.

Notable among these is the compound 5-(4-{2-[(6-methoxypyrimydin-4-yl)-methyl-amino]-ethoxyl}-benzyl)-thiazolidin-2,4-dione (hereinafter referred to as Compound I), described in that application in the form of a free base. Compound I in free base form presents problems of stability and solubility what do not permit it to be purified and handled suitably.

The bibliography contains a description (WO 9405659) of an improvement in the aqueous stability and solid-form stability of thiazolidinediones of structure similar to that of Compound I, by means of formation of the corresponding salts of acids, preferably of maleic acid.

However, Compound I does not form salts with acids such as tartaric or citric acid, and its corresponding salts with hydrochloric and maleic acid do not possess desirable aqueous solubility, nor good stability of said solution.

Surprisingly, the authors of this invention have found a new salt of Compound I which is of high aqueous solubility (higher than 1 mg/ml) and good stability. Advantageously, the new salt object of this invention permits its purification without problems of hygroscopicity or formation of solvates, which characteristics provide it with significant advantages for its industrial formulation and use. The new salt also shows a better oral absorption profile than the free base.

### DESCRIPTION OF THE INVENTION

The object of this invention is the sodium salt of 5-(4-{2-[(6-methoxy-pyrimydin-4-yl)-methyl-amino]-ethoxy}-benzyl)-thiazolidin-2,4-dione (hereinafter referred to as Sodium Salt).

Also object of this invention are three polymorphic forms of the Sodium Salt, which are disclosed below.

a) A polymorphic form of the Sodium Salt (hereinafter called Polymorph I) characterised in that it presents a X-ray powder diffractogram using Cu Kα radiation in accordance with FIG. 4. The positions of several significant peaks of said diffractogram are presented in Table 1.

Polymorph I provides an IR spectrum which presents the following characteristic bands at 3009, 2990, 2915 and 2904 nm, and of weak intensity at 1427, 1226, 1026, 553 nm (see FIG. 1).

TABLE 1

| | d value | |
|---|---|---|
| Angle 2θ [°] | [Å] | Hkl indices |
| 3.16 ± 0.10 | 28.0 ± 0.1 | 0 0 1 |
| 6.31 ± 0.05 | 14.01 ± 0.05 | 0 0 2 |
| 9.47 ± 0.05 | 9.33 ± 0.05 | 0 0 3 |
| 15.78 ± 0.05 | 5.61 ± 0.05 | 1 1 0 |
| 18.19 ± 0.05 | 4.87 ± 0.05 | 0 1 4 |
| 19.39 ± 0.05 | 4.57 ± 0.05 | 2 0 −3 |
| 20.68 ± 0.05 | 4.29 ± 0.05 | 1 1 4 |
| 22.47 ± 0.05 | 3.96 ± 0.05 | 2 1 1 |
| 29.92 ± 0.05 | 2.98 ± 0.05 | 2 0 −8 |

b) A polymorphic form of the Sodium Salt (hereinafter called Polymorph II) characterised in that it provides a X-ray powder diffractogram using Cu Kα radiation in accordance with FIG. 5. The positions of several significant peaks of said diffractogram are presented in Table 2.

TABLE 2

| Angle 2θ [°] | d value [Å] |
|---|---|
| 2.96 ± 0.10 | 29.9 ± 0.1 |
| 5.92 ± 0.05 | 14.93 ± 0.05 |
| 8.87 ± 0.05 | 9.97 ± 0.05 |
| 13.58 ± 0.05 | 6.52 ± 0.05 |
| 15.95 ± 0.05 | 5.55 ± 0.05 |
| 16.41 ± 0.05 | 5.40 ± 0.05 |
| 21.55 ± 0.05 | 4.12 ± 0.05 |
| 26.13 ± 0.05 | 3.41 ± 0.05 |

c) A polymorphic form of the Sodium Salt (hereinafter called Polymorph III) characterised in that it provides a X-ray powder diffractogram using Cu Kα radiation in accordance with FIG. 6. The positions of several significant peaks of said diffractogram are presented in Table 3.

TABLE 3

| Angle 2θ [°] | d value [Å] |
|---|---|
| 3.14 ± 0.10 | 28.1 ± 0.1 |
| 6.25 ± 0.05 | 14.13 ± 0.05 |
| 9.40 ± 0.05 | 9.41 ± 0.05 |
| 14.43 ± 0.05 | 6.13 ± 0.05 |
| 15.79 ± 0.05 | 5.61 ± 0.05 |
| 16.52 ± 0.05 | 5.36 ± 0.05 |
| 18.05 ± 0.05 | 4.91 ± 0.05 |

The IR spectra of Polymorphs II (see FIG. 2) and III (see FIG. 3) clearly show differences between the intensities of the bands between 1200-1185 nm and 570-550 nm (see FIGS. 10 and 11). Despite the fact that small differences in the spectra can be discerned, the IR technique is not very precise for distinguishing the Polymorphs II and III from each other, although it does permit these two polymorphs to be distinguished from Polymorph I.

Polymorph I is monoclinic. The organic anion has a chiral centre and both enantiomers are present in Polymorph I. The sodium cation is surrounded by four oxygen atoms, two nitrogen atoms and one sulphur atom belonging to the 1,3-thiazolidin-2,4-dione fragment of five anions. With two of them it forms four-member chelates through the nitrogen and one oxygen. The coordination polyhedron of the sodium is a highly distorted pentagonal bipyramid. The ions are arranged in a crystal in the form of layers parallel to the plane (001). The centre of the layers is made up of the sodium cations surrounded by the 1,3-thiazolidin-2,4-dione fragments. The tails of the anions are removed to either side of this central part (see FIG. 8).

Also object of this invention is a method for preparing the Sodium Salt. The Sodium Salt can be prepared by causing 5-(4-{2-[(6-methoxy-pyrimydin-4-yl)-methyl-amino]-ethoxy}-benzyl)-thiazolidin-2,4-dione to react with a source of sodium ion (Na⁺) of base character, such as sodium hydroxide, sodium alkoxide, sodium hydride, in a suitable solvent.

Also object of this invention is a method for preparing the Polymorph I. Polymorph I can be prepared by precipitation or by crystallisation. Thus, a method for preparing Polymorph I according to the invention comprises:

a) preparing a solution of the Sodium Salt, in an organic solvent or in a mixture of solvents, under reflux, and cooling to room temperature, or

b) preparing a saturated solution of the Sodium Salt at room temperature in methyl or ethyl alcohol and cooling to a temperature lower than room temperature, or

c) preparing a solution of the Sodium Salt in water or methyl alcohol and pouring it into an insolubilising solution, or

d) causing a solution of 5-(4-{2-[(6-methoxy-pyrimydin-4-yl)-methyl-amino]-ethoxy}-benzyl)-thiazolidin-2,4-dione in isopropanol to react under reflux with a source of sodium ion of base character, preferably sodium hydroxide, and cooling to a temperature lower than room temperature.

and then isolating the polymorphic form of the solvent.

Also object of this invention is a method for making Polymorph II. Polymorph II can be prepared by evaporation. Thus, a method for preparing Polymorph II according to the invention comprises:

a) preparing a solution of the Sodium Salt in water or in an alcohol and eliminating the solvent by evaporation at atmospheric pressure, at room temperature, or

b) preparing a solution of the Sodium Salt in an alcohol and eliminating the solvent by evaporation at low pressure and within a temperature range of 30-80° C.

Also object of this invention is a method for making Polymorph III. polymorph III can be prepared by evaporation of an aqueous solution. Thus, a method for preparing Polymorph III according to the invention comprises preparing a solution of the Sodium Salt in water and eliminating the solvent at low pressure and within a temperature range of 40-80° C.

The Compound (I) is prepared as described in Spanish patent application no. 9902533, whose content is incorporated herein by way of reference.

The compounds object of this invention present hyperglemic and hyperlipidic activity.

The invention thus provides the Sodium Salt and its polymorphic forms called Polymorphs I, II and III for use as a therapeutically active substance, and in particular for use in the treatment and/or prophylaxis of hyperglicemia and/or hyperlipidemia and/or for use in the treatment of complications associated with resistance to insulin, such as hypertension, hyperuricemia or other cardiovascular, metabolic and endocrine disorders.

The compounds object of this invention can be used alone or in combination with one or more antidiabetic agents such as the sulfonylureas, biguanides, alpha glucosidase inhibitors, beta agonists or insulin.

Thus, under another aspect, this invention provides the Sodium Salt and the polymorphic forms thereof called Polymorph I, II and III, alone or in combination with one or more antidiabetic agents, for the manufacture of a medicine for the treatment and/or prophylaxis of hyperglycemia and/or hyperlipidemia and/or for the treatment of complications associated with resistance to insulin, such as hypertension, hyperuricemia or other cardiovascular, metabolic and endocrinal disorders.

The compounds object of this invention can be administered as they are or, preferably, as a pharmaceutical composition which includes at least one pharmaceutically acceptable excipient.

In accordance with this, this invention provides a pharmaceutical composition which includes the Sodium Salt and the polymorphic forms thereof named Polymorphs I, II and III, and a therapeutically active and suitable quantity of at least once excipient.

The compositions provided by this invention can be administered by any appropriate via, but preferably orally or parenterally.

The compositions for parenteral or topical administration can be injectable solutions, infusions, suppositories or transdermic systems. The pharmaceutical compositions for oral administration can be solid, such as tablets or capsules prepared by the conventional means with pharmaceutically acceptable excipients, or liquids such as aqueous or oleous solutions, syrups, elixirs, emulsions or suspensions prepared by the conventional means with pharmaceutically acceptable additives.

Tablets and capsules are the preferred forms of administration.

In accordance with conventional pharmaceutical practice, the excipients can include diluents, disintegrators, wetting agents, lubricants, colorants, flavourings or other conventional adjuvants.

Typical excipients include, for example, microcrystalline cellulose, starch, polyvinyl pyrrolidone, magnesium stearate or sodium lauryl sulphate.

### DESCRIPTION OF THE FIGURES

FIG. 1 shows the IR spectrum of Polymorph I. The y-axis shows the percentage of transmittance and the x-axis the frequency expressed in cm⁻¹.

FIG. 2 show the IR spectrum of Polymorph II.

FIG. 3 show the IR spectrum of Polymorph III.

FIG. 4 shows the X-ray powder diffractogram of Polymorph I. The y-axis shows the counts and the x-axis angle 2 Theta.

FIG. 4 shows the X-ray powder diffractogram of Polymorph II.

FIG. 5 shows the X-ray powder diffractogram of Polymorph II.

FIG. 6 shows the X-ray powder diffractogram of Polymorph III.

FIG. 7 shows the three X-ray diffractograms of Polymorphs I, II and III, respectively, in order to facilitate comparison thereof, where PI indicates Polymorph I, P II Polymorph II and P III Polymorph III.

FIG. 8 shows the contents of the elemental cell of Polymorph I.

FIG. 9 shows an enlargement of the IR spectrum of Polymorph I, of the zone included between 2700 and 3150 cm⁻¹.

FIG. 10 shows an enlargement of the IR spectrum of Polymorph II, of the zone included between 2700 and 3150 cm⁻¹.

FIG. 11 shows an enlargement of the IR spectrum of Polymorph III, of the zone included between 2700 and 3150 cm⁻¹.

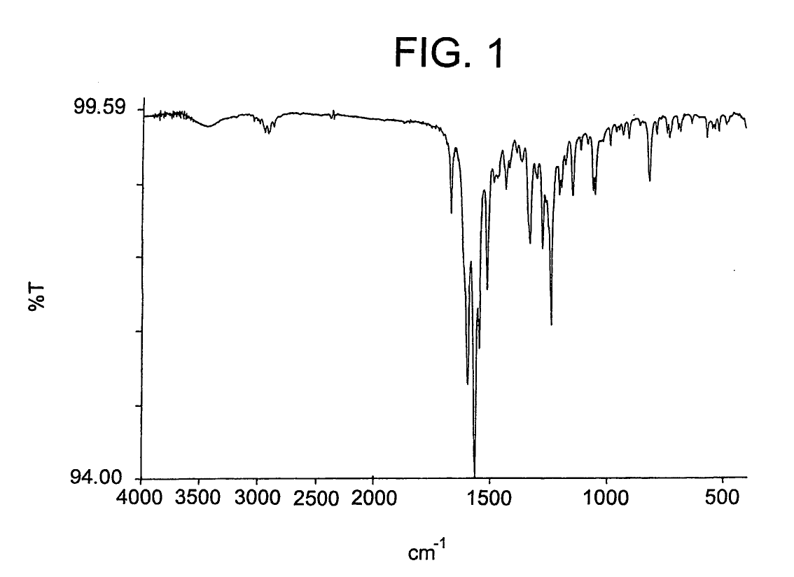

### EXPERIMENTAL PART

Below, by way of non-restrictive explanation of the invention, is an outline of the following examples.

### EXAMPLES OF SYNTHESIS

### Example 1

Sodium Salt of 5-(4-(2-(6-methoxy-pyrimydin-4-yl) amino) ethoxy) benzyl) thiazolidin-2,4-dione

To a suspension of 12.0 g of 5-(4-(2-(6-methoxy-pyrimydin-4-yl) amino)ethoxy)benzyl)thiazolidin-2,4-dione in 60 ml of 95% EtOH is added drop by drop a solution of 1.4 g of NaOH in a mixture of 6.0 ml of 95% EtOH and 3.6 ml of water. Once addition is completed, the mixture is stirred for 2 hours at room temperature.

The mixture is cooled to 0-5° C., stirred for one hour and filtered. The solid is dried in an oven at 40° C. 11.5 g of the product of the title is obtained. Yield: 90.8%.

Most of the product obtained corresponds to Polymorph I.

¹H-NMR spectrum (200 MHz, D₂O, δ ppm, TMS): 8.0 (s, 1H, pirimidine)/7,0 (d, 2H, bencenic ring)/6,65 (d, 2H, bencenic ring)/5,6 (s, 1H, pirimidine)/4,4 (d×d, 1H, thiazolidindione)/4,0 (sc, 2H, CH₂O)/3,7 (sc, 2H, NCH₂)/3,7 (s, 3H, OCH₃)/3,2 (d×d, 1H, CH₂ bridge)/2,85 (s, 3H, NCH₃)/2,8 (d×d, 1H, CH₂ bridge).

### Example 2

Sodium Salt of 5-(4-(2-(6-methoxypyrimydin-4-yl) amino)ethoxy)benzyl)thiazolidin-2,4-dione (Polymorph I)

11.5 g of the product obtained in example 1 is suspended in 46 ml of IPA. The mixture is stirred and heated under reflux. Water is then added drop by drop until dissolution (12 ml). The heating is turned off and the mixture is stirred for a few hours. It is cooled to 0-5° C. It is stirred for one hour and filtered. The solid is dried in an oven at 40° C. 9.7 g of the product of the title is obtained. Recryst. yield: 84.3%.

Melting point: decomposition at approx. 240° C.

IR spectrum (KBr) (Polymorph I): 3000-3050 (t CH ar.)/2900-3000 (t CH al.)/1670, 1600 (t C═N)/1560 (t C═O)/1540, 1510 (t C═C ar.)/1230 (t C—O).

X-ray spectrum: coincides with the diffractogram of Polymorph I.

### Example 3

Sodium Salt of 5-(4-(2-(6-methoxypyrimydin-4-yl) amino)ethoxy)benzyl)thiazolidin-2,4-dione (Polymorph I)

0.1 g of the product obtained in Example 1 is dissolved in 3 ml of water. The solution is poured all at once, with agitation and at room temperature, onto 30 ml of acetone.

It is left to rest. It is filtered and the precipitated product is dried to obtain the product of the title.

X-ray spectrum: coincides with the diffractogram of Polymorph I.

### Examples 4-8

Sodium Salt of 5-(4-(2-(6-methoxypyrimydin-4-yl) amino)ethoxy)benzyl)thiazolidin-2,4-dione (Polymorph I)

0.1-0.3 g of the product obtained in Example 1 is dissolved in 10 ml of ethanol. The solution is poured all at once, with agitation and at room temperature onto 100 ml of the solvents indicated below:

| EXAMPLE | Solvent |
|---|---|
| 4 | Tetrahydrofuran |
| 5 | Acetone |
| 6 | Ethyl acetate |
| 7 | Chloroform |
| 8 | Toluene |

It is left to rest. It is filtered and the precipitated product is dried to obtain the product of the title.

X-ray spectrum: the diffractogram of Polymorph (I) is obtained in all cases.

### Examples 9-19

Sodium Salt of 5-(4-(2-(6-methoxypyrimydin-4-yl) amino)ethoxy)benzyl)thiazolidin-2,4-dione (Polymorph I)

The product obtained in Example 1 is dissolved in a solvent under reflux. The resulting solution is left to cool slowly with stirring to room temperature. The solid obtained is filtered and dried to obtain the product of the title.

The table which follows shows the amounts of the product of Example 1 used, together with the volume and the solvent or mixture of solvents used.

| | Quantity_{Example 1} | | |
|---|---|---|---|
| EXAMPLE | (g) | Solvent (s) | Vₛₒₗᵥₑₙₜ (ml) |
| 9 | 0.52 | Methanol | 20 |
| 10 | 0.48 | Ethanol | 124 |
| 11 | 0.32 | Isopropyl alcohol | 232 |
| 12 | 0.41 | Water:Acetone | 1.2:10 |
| 13 | 1.51 | Water:Isopropyl | 3.5:20 |
| | | alcohol | |
| 14 | 0.40 | Methanol:Acetone | 15:20 |
| 15 | 0.50 | Methanol:Ethyl | 20:20 |
| | | Acetate | |
| 16 | 0.16 | Ethanol:Acetone | 15:15 |
| 17 | 0.17 | Ethanol:Ethyl | 37:37 |
| | | Acetate | |
| 18 | 0.21 | Ethanol:THF | 31:31 |
| 19 | 0.40 | Ethanol:Toluene | 73:20 |

X-spectrum: the diffractogram of Polymorph (I) is obtained in all cases.

### Example 20

Sodium Salt of 5-(4-(2-(6-methoxypyrimydin-4-yl) amino)ethoxy)benzyl)thiazolidin-2,4-dione (Polymorph I)

A saturated solution of the product obtained in Example 1 in ethanol is prepared.

The solution is left to cool to 2° C.

After 48 hours the crystallised product is filtered and dried to obtain the product of the title.

X-ray spectrum: coincides with the diffractogram of Polymorph I.

### Example 21

Sodium Salt of 5-(4-(2-(6-methoxypyrimydin-4-yl) amino)ethoxy)benzyl)thiazolidin-2,4-dione (Polymorph I)

A saturated solution of the product obtained in Example 1 in methanol is prepared.

The solution is left to cool to 2° C.

After 48 hours the crystallised product is filtered and dried to obtain the product of the title.

X-ray spectrum: coincides with the diffractogram of Polymorph I.

### example 22

Sodium Salt of 5-(4-(2-(6-methoxypyrimydin-4-yl) amino)ethoxy)benzyl)thiazolidin-2,4-dione (Polymorph I)

A saturated solution of the product obtained in Example 1 in ethanol is prepared.

The solution is left to cool to −3° C.

After 48 hours the crystallised product is filtered and dried to obtain the product of the title.

X-ray spectrum: coincides with the diffractogram of Polymorph I.

### Example 23

Sodium Salt of 5-(4-(2-(6-methoxypyrimydin-4-yl) amino)ethoxy)benzyl)thiazolidin-2,4-dione (Polymorph I)

12.0 g of 5-(4-(2-(6-methoxypyrimydin-4-yl) amino)ethoxy)benzyl)thiazolidin-2,4-dione is suspended in 48 ml of isopropanol. The mixture is agitated and heated under reflux. A solution of 1.36 g of NaOH in 12 ml of water is added drop by drop. Once the addition is completed, 2 ml of water is added drop by drop. The suspension then changes to a solution. The heating is turned off. The mixture is agitated until it reaches room temperature, during which time it is turned once again into a suspension. It is then cooled to 0-5° C., agitated for one hour and filtered. The solid is dried in an oven at 40° C. 9.9 g of the product is obtained.

Yield: 78.1%.

Melting point: decomposition at approx. 240° C.

IR spectrum (KBr) (Polymorph I): 3000-3050 (t CH ar.)/2900-3000 (t CH al.)/1670, 1600 (t C═N)/1560 (t C═O)/1540, 1510 (t C═C ar.)/1230 (t C—O).

X-ray spectrum: coincides with the diffractogram of Polymorph I.

### Example 24

Sodium Salt of 5-(4-(2-(6-methoxypyrimydin-4-yl) amino)ethoxy)benzyl)thiazolidin-2,4-dione (Polymorph II)

0.15 g of the product obtained in Example 1 is dissolved in 5 ml of water.

The solvent is evaporated at room temperature in crystallisation capsules to obtain the product of the title.

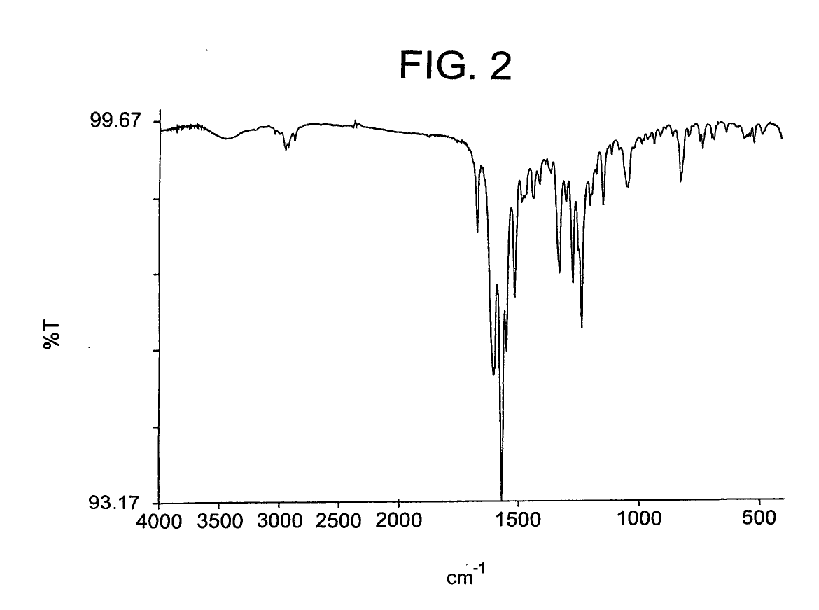

IR spectrum (KBr): coincides with FIG. 2.

X-ray spectrum: coincides with the diffractogram of Polymorph II.

### Example 25

Sodium Salt of 5-(4-(2-(6-methoxypyrimydin-4-yl) amino)ethoxy)benzyl)thiazolidin-2,4-dione (Polymorph II)

0.15 g of the product obtained in Example 1 is dissolved in 20 ml of methanol.

The solvent is evaporated at room temperature in crystallisation capsules to obtain the product of the title.

X-ray spectrum: coincides with the diffractogram of Polymorph II.

### Example 26

Sodium Salt of 5-(4-(2-(6-methoxypyrimydin-4-yl) amino)ethoxy)benzyl)thiazolidin-2,4-dione (Polymorph II)

0.15 g of the product obtained in Example 1 is dissolved in 180 ml of ethanol.

The solvent is evaporated at room temperature in crystallisation capsules to obtain the product of the title.

X-ray spectrum: coincides with the diffractogram of Polymorph II.

### Example 27

Sodium Salt of 5-(4-(2-(6-methoxypyrimydin-4-yl) amino)ethoxy)benzyl)thiazolidin-2,4-dione (Polymorph II)

0.5 g of the product obtained in Example 1 is dissolved in 50 ml of methanol.

The solvent is eliminated at low pressure, keeping the temperature of the bath at 50° C. to obtain the product of the title.

X-ray spectrum: coincides with the diffractogram of Polymorph II.

### Example 28

Sodium Salt of 5-(4-(2-(6-methoxypyrimydin-4-yl) amino)ethoxy)benzyl)thiazolidin-2,4-dione (Polymorph II)

0.5 g of the product obtained in Example 1 is dissolved in 500 ml of ethanol.

The solvent is eliminated at low pressure, keeping the temperature of the bath at 50° C. to obtain the product of the title.

X-ray spectrum: coincides with the diffractogram of Polymorph II.

### Example 29

Sodium Salt of 5-(4-(2-(6-methoxypyrimydin-4-yl) amino)ethoxy)benzyl)thiazolidin-2,4-dione (Polymorph III)

0.5 g of the product obtained in Example 1 is dissolved in 0.5 ml of water.

The solvent is eliminated at low pressure, keeping the temperature of the bath at 70° C. to obtain the product of the title.

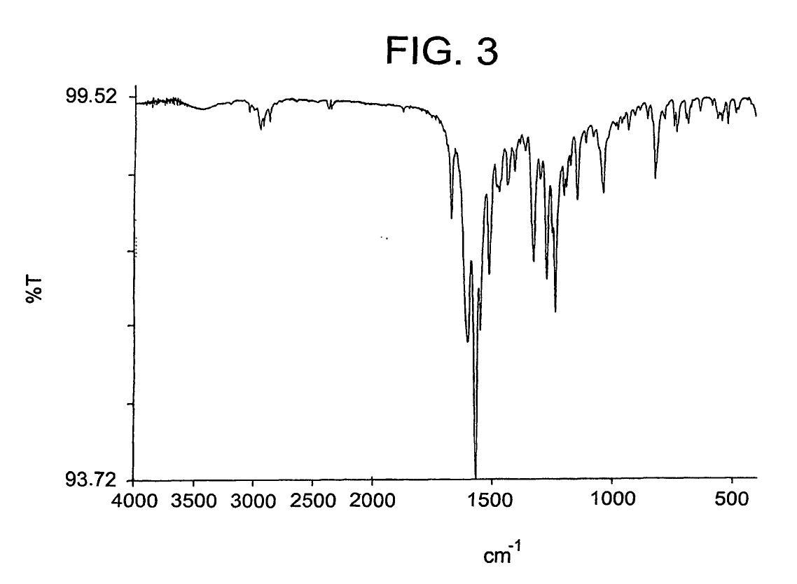

IR spectrum (KBr): coincides with FIG. 3.

X-ray spectrum: coincides with the diffractogram of Polymorph III.

1. Sodium salt of 5-(4-{2-[(6-methoxy-pyrimydin-4-yl)-methyl-amino]-ethoxy}-benzyl)-thiazolidin-2,4-dione.

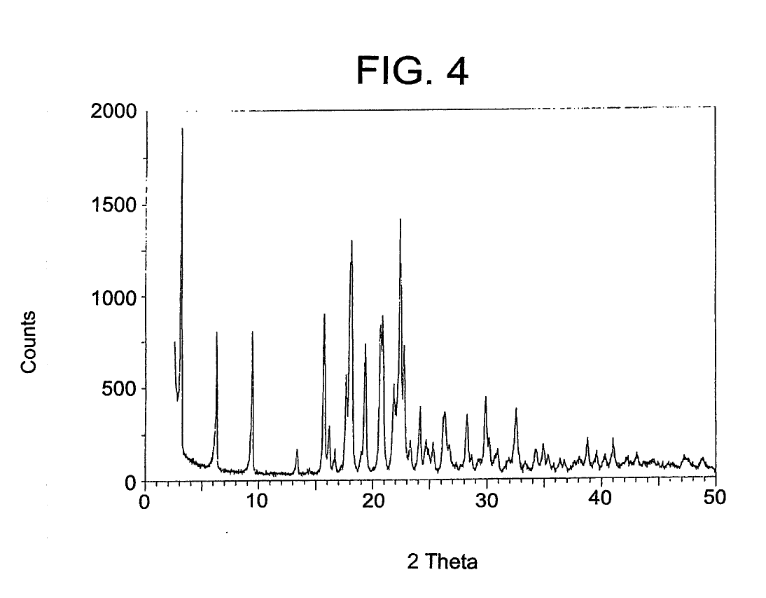

2. Polymorphic form of the compound as claimed in claim 1, characterised in that its X-ray powder diffractogram is shown in FIG. 4.

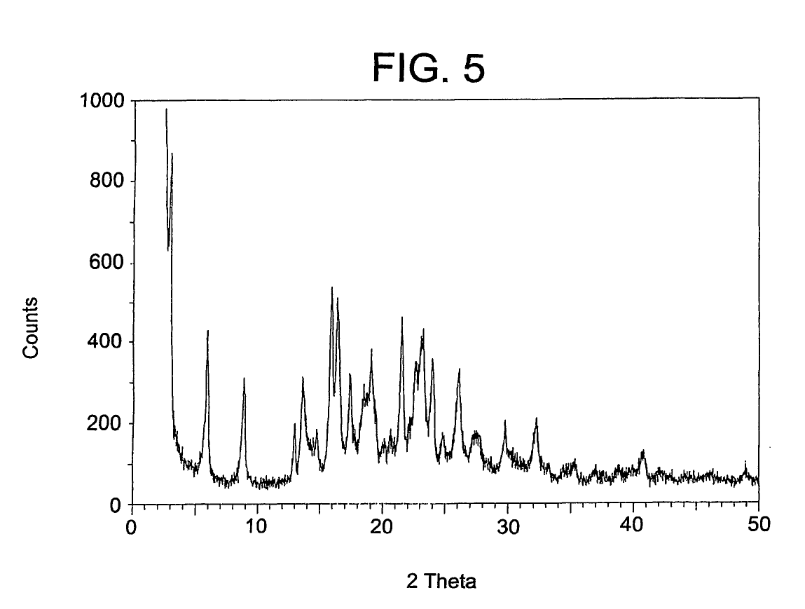

3. Polymorphic form of the compound as claimed in claim 1, characterised in that its X-ray powder diffractogram is shown in FIG. 5.

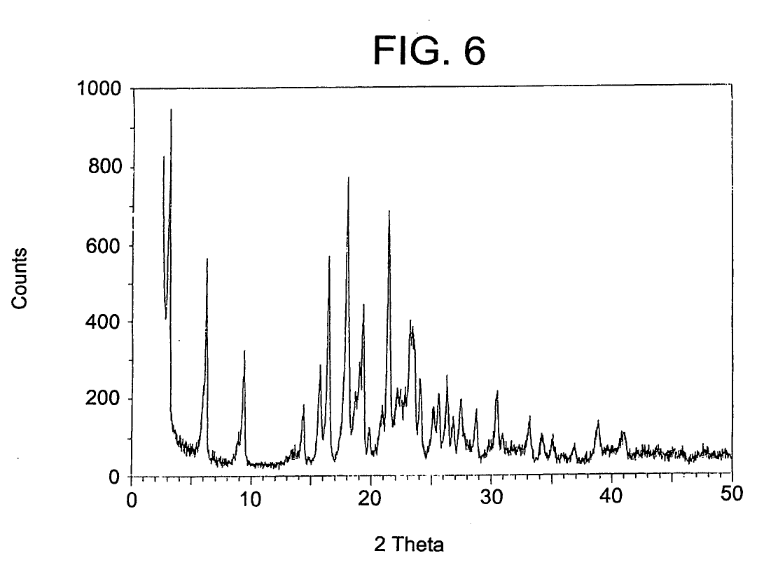

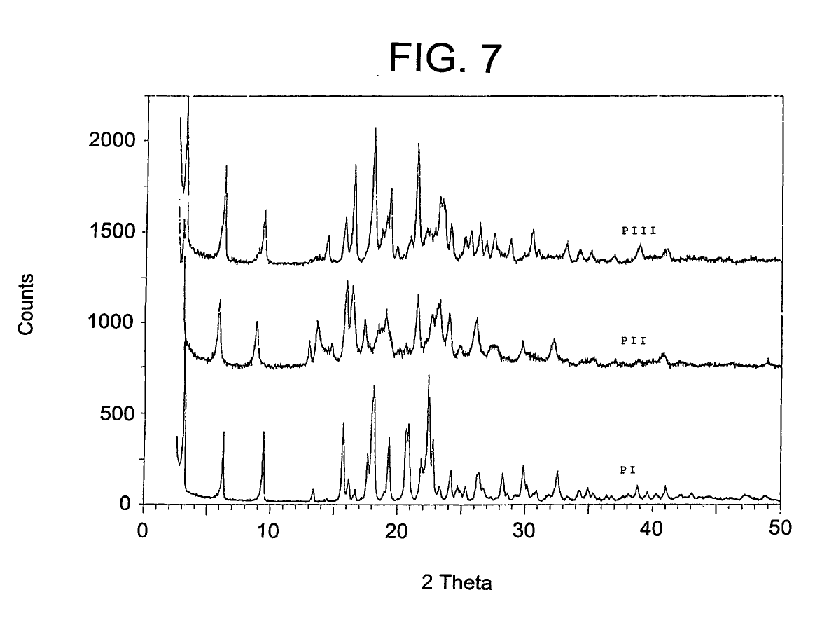

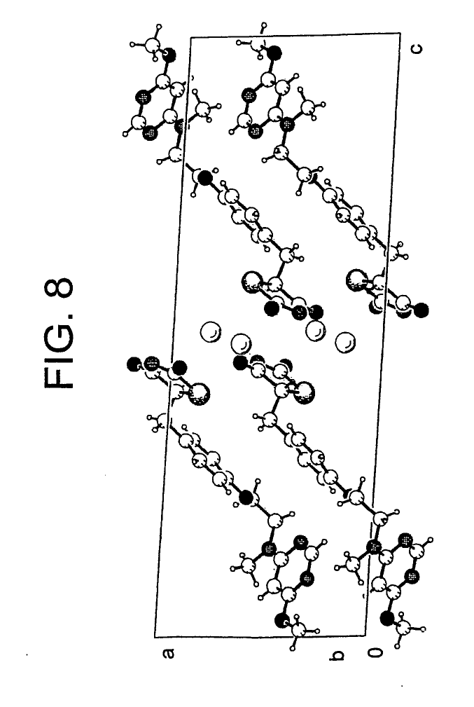

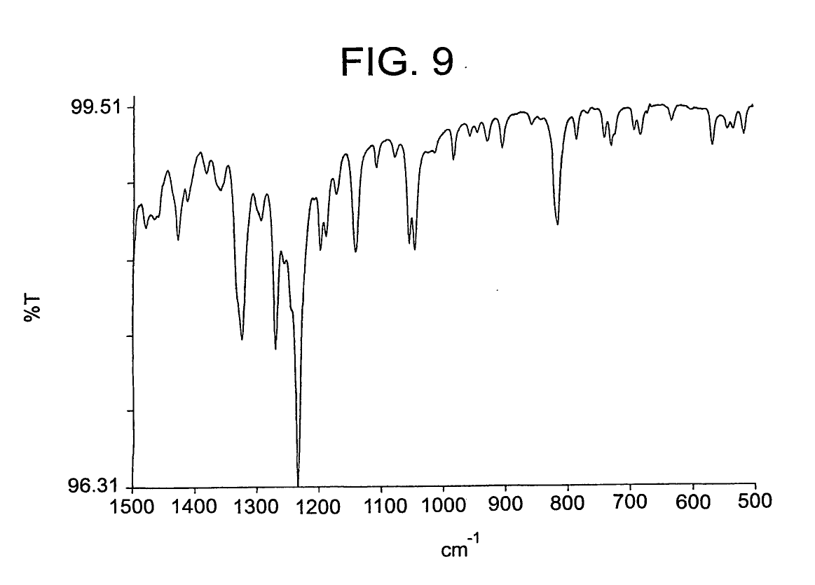

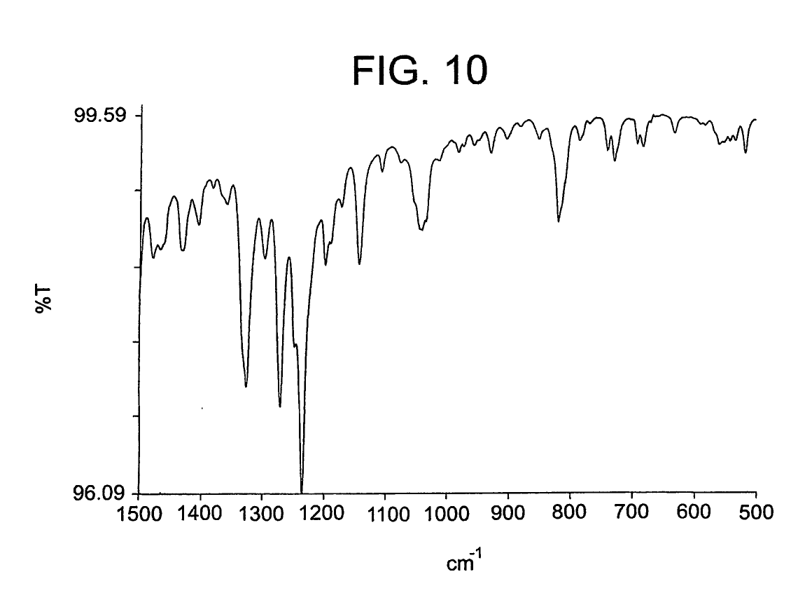

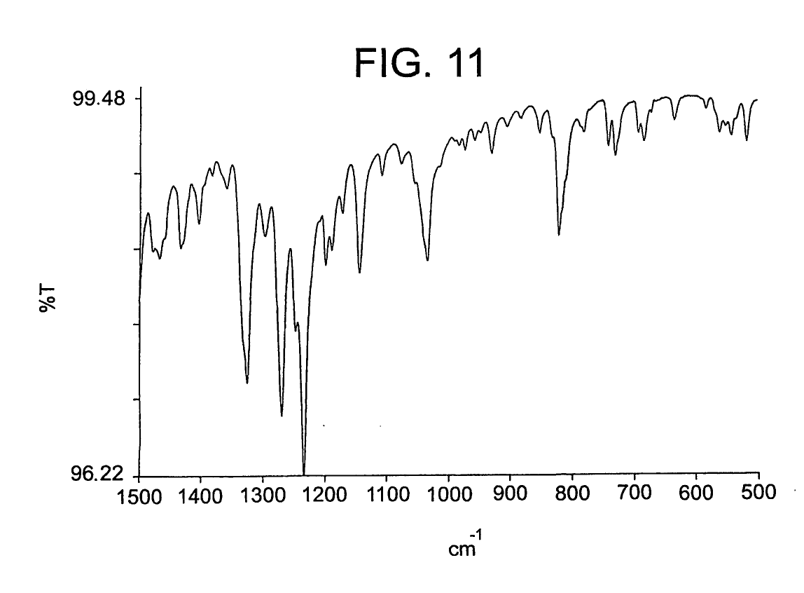

4. Polymorphic form of the compound as claimed in claim 1, characterised in that its X-ray powder diffractogram is shown in FIG. 6.

5. Method for preparing the compound as claimed in claim 1, characterised in that it includes causing 5-(4-{2-[(6-methoxy-pyrimydin-4-yl)-methyl-amino]-ethoxy}-benzyl)-thiazolidin-2,4-dione to react with a source of sodium ion (Na⁺) of base character.

6. Method as claimed in claim 5, characterised in that said source of sodium ion is sodium hydroxide, sodium alkoxide or sodium hydride.

7. Method for making a compound as claimed in claim 2, characterised in that it comprises:
a) preparing a solution of the compound as claimed in claim 1, in an organic solvent or in a mixture of solvents, under reflux, and cooling to room temperature, or
b) preparing a saturated solution of the compound as claimed in claim 1, at room temperature in methyl or ethyl alcohol and cooling to a temperature lower than room temperature, or
c) preparing a solution of the compound as claimed in claim 1, in water or methyl alcohol and pouring it into an insolubilising solution, or
d) causing a solution of 5-(4-{2-[(6-methoxy-pyrimydin-4-yl)-methyl-amino]-ethoxy}-benzyl)-thiazolidin-2,4-dione in isopropanol to react under reflux with a source of sodium ion of base character, preferably sodium hydroxide, and cooling slowly to a temperature lower than room temperature, and, then, recovering the polymorphic form of the solvent.

8. Method for making a compound as claimed in claim 3, characterised in that it comprises:
a) preparing a solution of the compound as claimed in claim 1, in water or in an alcohol, and eliminating the solvent by evaporation at atmospheric pressure, at room temperature, or
b) preparing a solution of the compound as claimed in claim 1, in an alcohol, and eliminating the solvent by evaporation at low pressure and within a temperature range of 30-80° C.

9. Method for making a compound as claimed in claim 4, characterised in that it comprises preparing a solution of the compound as claimed in claim 1 in water and eliminating the solvent at low pressure and within a temperature range of 40-80° C.

10. Pharmaceutical composition which includes a compound as defined in any of claims 1 to 4, in a therapeutically active quantity and a suitable quantity of at least one excipient.

11. Compound as defined in any of claims 1 to 4 for use as an antihyperglycemic agent and/or an antihyperlipidemic agent and/or an insulin sensitizer.

12. Utilisation of a compound as defined in any of claims 1 to 4, alone or in combination with one or more antidiabetic agents such as the sulfonylureas, biguanides, alpha glucosidase inhibitors, beta agonists or insulin, for the manufacture of a medicament for treating and/or prophylaxis of hyperglycemia and/or hyperlipidemia and/or for treating complications associated with resistance to insulin, such as hypertension, hyperuricemia or other cardiovascular, metabolic and endocrinal disorders.
